(19) United States
(12) Patent Application Publication
(10) Pub. No.: US 2015/0064164 A1
(43) Pub. Date: Mar. 5, 2015

(54) TISSUE DISRUPTION TREATMENT AND COMPOSITION FOR USE THEREOF

(71) Applicant: Cambridge Scientific Pty Ltd, Leederville (AU)

(72) Inventors: Jeffrey D. Edwards, Claremont (AU); Maud Louise Eijkenboom, Melville (AU)

(21) Appl. No.: 14/491,862

(22) Filed: Sep. 19, 2014

Related U.S. Application Data
(63) Continuation of application No. 11/218,382, filed on Sep. 1, 2005, now abandoned.

(51) Int. Cl. A61K 47/02; C12P 21/06; A61K 35/16

(52) U.S. Cl. CPC A61K 47/02; A61K 35/16; C12P 21/06
USPC 424/94.64; 424/530; 424/531; 435/68.1

(57) ABSTRACT

The present invention relates to an agent having activity in the treatment of a tissue disruption. In particular the present invention relates to a composition comprising an effective amount of an active fraction having tissue healing properties

### FIELD OF THE INVENTION

The present invention relates to an agent having activity in the treatment of a tissue disruption. In particular the present invention relates to a composition comprising an effective amount of an active fraction having tissue healing properties, wherein said active fraction is separated from a mixture of plasma and/or serum and at least one metal, metal ion or metal salt thereof and wherein said mixture has been denatured.

### BACKGROUND OF THE INVENTION

The treatment of tissue disruptions such as sunburn, soft and connective tissue injury and wounds can be impeded by the lack of effective therapeutics. Part of the problem is a lack of understanding of the process of healing.

Wound healing is usually a coordinated, stereotyped sequence of events that includes (a) tissue disruption and loss of normal tissue architecture; (b) cell necrosis and haemorrhage; hemostasis (clot formation); (c) infiltration of segmented and mononuclear inflammatory cells, with vascular congestion and tissue oedema; (d) dissolution of the clot as well as damaged cells and tissues by mononuclear cells (macrophages) (e) formation of granulation tissue (fibroplasia and angiogenesis). This sequence of cellular events has been observed in wounds from all tissues and organs generated in a large number of mammalian species (Gailet et al., 1994, Curr. Opin. Cell. Biol. 6:717-725). Therefore, the cellular sequence described above is a universal aspect of the repair of all mammalian tissues.

Many of the current treatment compositions for tissue disruptions have difficulties addressing the optimum requirements. For example, with respect the treatment of wounds (one type of tissue disruption) the optimum requirements are acceleration of the rate of wound contraction, increasing the rate of epithelialisation and increasing the rate of maturation of granulation material, thereby ultimately reducing the time to full maturity of the healed wound.

Similar problems have also been experienced with other types of tissue disruptions. For example, burns have been unsuccessfully treated. With respect to deep soft tissue injuries, previous treatments have included injections of various materials to repair or swell soft tissues. Some of the agents used include liquid silicone, collagen in various forms such as chemically cross-linked and fibrous forms and hyaluronic acid.

Unfortunately, none of these procedures or materials are considered to be ideal owing to short-comings in effectiveness or efficacy. For example, liquid silicone was banned by the FDA when it was discovered that it could migrate to distant parts of the body and cause physiological and clinical problems.

It is therefore desirable to have a treatment composition that can be used to treat all types of tissue disruptions including soft and connective tissue injuries, deep tissue injuries, surface wounds and open wounds, wherein the time to full maturity of the injury is reduced by halting primary as well as secondary damage, and accelerating the rate of tissue repair.

Following extensive biochemical laboratory research, the present inventors have developed a composition capable of overcoming or at least alleviating some of the problems associated with prior art tissue disruption treatments.

### SUMMARY OF THE INVENTION

In a first aspect, the present invention provides a composition comprising an effective amount of an active fraction separated from a mixture of plasma and/or serum and at least one metal, metal ion or metal salt thereof, wherein said mixture has been denatured and is effective in healing tissue disruptions.

The plasma or serum may be obtained from any animal source. Preferably, the plasma or serum is isolated from an animal selected from the group consisting of human, equine, bovine, ovine, murine, caprine and canine.

In one embodiment, the plasma and/or serum is dried and lyophilised before use.

Once the plasma and/or serum has been obtained it is mixed with at least one metal, metal ion or metal salt thereof. The metal, metal ion or metal salt thereof can be any metal. In one embodiment, the metal is selected from the group consisting of nickel, sodium, copper, zinc, cobalt, iron, magnesium, manganese, potassium, silver and mercury, ions or salts thereof and mixtures thereof.

Once the metal, metal ion or metal salt thereof has been mixed with the plasma and/or serum, it is preferably heated to at least 50° C. Preferably, the mixture is heated to about 65° C.

In some embodiments, a protease such as trypsin is preferably added before heating or after heating. At which point the resultant mixture is again heated then allowed to cool to produce a mixture that is capable of healing tissue disruptions such as soft and connective tissue injuries and wounds.

The second heating step is preferably carried out between about 80° C. and about 150° C., more preferably between about 90° C. and about 130° C. and most preferably, about 120° C.

The wound healing mixture of the present invention can be used directly or further separated to produce a fraction having healing properties.

The composition of present invention can comprise at least a fraction of a mixture as described above. More preferably, the composition of present invention is optionally admixed with a pharmaceutical carrier. Any pharmaceutical carrier known in the art may be used.

Accordingly, in a second aspect the present invention provides a composition obtained by:

(a) heat denaturing a mixture of plasma and/or serum and at least one metal, metal ion or metal salt thereof; and

(b) separating an active fraction from said denatured mixture;

wherein said active fraction is capable of healing tissue disruptions.

The step of separating the active fraction can be by chromatography such as affinity chromatography, column chromatography, partition chromatography, gel-filtration chromatography with a suitable solvent or solvent mixture.

In some embodiments, the method further comprises the steps of incubating said mixture in the presence of a protease to produce a digested mixture; and heating said digested mixture. These steps can be undertaken before or after addition of the at least one metal, metal ion or metal salt.

Accordingly, in a third aspect the present invention provides a composition obtained by:

(a) heat denaturing a mixture of plasma and/or serum and at least one metal, metal ion or metal salt thereof;

(b) incubating said mixture in the presence of a protease to produce a digested mixture;

(c) heating said digested mixture; and

(d) separating an active fraction from said denatured mixture;

wherein said active fraction is capable of healing tissue disruptions.

The step of separating the active fraction can be by chromatography such as affinity chromatography, column chromatography, partition chromatography, gel-filtration chromatography with a suitable solvent or solvent mixture.

In some embodiments, steps (b) and (c) are performed before the addition of at least one metal, metal ion or metal salt thereof. In further embodiments, step (a) further comprises the addition of NaHCO₃.

The step of denaturing the mixture by heat can be carried out at a temperature greater than 65° C.

The fractionation step (d) can be performed by chromatography on a polyamide column; however, any other method of fractionation may be used.

In a fourth aspect, the present invention provides a method for providing treatment of a tissue disruption in a subject, said method comprising administering to the subject an effective amount of a composition comprising an effective amount an active fraction separated from a mixture of plasma and/or serum and at least one metal, metal ion or metal salt thereof, wherein said mixture has been denatured and wherein said active fraction is capable of healing tissue disruptions.

The method of administration may be any method known in the art. In some embodiments, the composition is administered topically, systemically, intramuscularly, subcutaneously, intraperitoneally, intrapleurally, intraarticularly, intrathecally, rectally, vaginally, or by inhalation. Most preferably, the composition is administered topically.

In a fifth aspect, the present invention provides a composition for treating tissue disruptions in a subject comprising a pharmaceutically acceptable carrier and an effective amount of an active fraction separated from a mixture of plasma and/or serum and at least one metal, metal ion or metal salt thereof, wherein said mixture has been denatured and wherein said active fraction is capable of healing tissue disruptions.

In a sixth aspect, the present invention provides a tissue disruption treatment substance which is extracted from a mixture of plasma and/or serum and at least one metal, metal ion or metal salt thereof, wherein said mixture has been denatured and wherein said substance is capable of healing tissue disruptions.

The tissue disruption treatment substance can be further admixed with a pharmaceutically acceptable carrier. The carrier can be at least one member selected from the group consisting of distilled water, physiologically saline solution, Ringer's solution, plant oil, synthetic fatty acid glycerides, higher fatty acid esters, propylene glycol, lactose, mannitol, corn starch, crystalline cellulose, gum arabicum, gelatin, potato starch, carmerose, carmerose calcium, talc, and magnesium stearate.

In a seventh aspect, the present invention provides a method of treating a tissue disruption in a subject afflicted thereof comprising the step of administering to a subject in need thereof a therapeutic amount of a composition comprising an active fraction separated from a mixture of plasma and/or serum and at least one metal, metal ion or salt thereof, wherein said mixture has been denatured and wherein said fraction is admixed with a pharmaceutically acceptable carrier.

The tissue disruption can be selected from the group consisting of a lesion, a wound, a microbial infection, a burn including sunburn, an ulcer, a soft or connective tissue injury including a tendon/ligament injury or an overuse injury, inflammation and a dermal condition. In some embodiments, the tissue disruption is a soft and/or connective tissue injury or a burn including sunburn.

In an eighth aspect the present invention provides a method of treating a tissue disruption comprising the step of applying to said disrupt tissue a therapeutic amount of a composition comprising an active fraction separated from a mixture of plasma and/or serum and at least one metal, metal ion or salt thereof, wherein said mixture has been denatured and wherein said fraction is admixed with a pharmaceutically acceptable carrier.

In a ninth aspect the present invention provides a wound dressing comprising an active fraction separated from a mixture of plasma and/or serum and at least one metal, metal ion or salt thereof, wherein said mixture has been denatured and wherein said dressing is capable of healing tissue disruptions.

It is further contemplated that the active fraction of plasma and/or serum and at least one metal, metal ion or salt thereof can also be used to coat medical devices used in the treatment of diseases or disorders. The medical devices that can be thus coated are, for example, catheters, guide channels, probes, cardiac valves, soft tissue replacements, replacements of animal origin, artificial tendons, bone and cardiovascular replacements, contact lenses, blood oxygenators, artificial kidneys, hearts, pancreas and livers, blood bags, syringes, surgical instruments, filtration systems, laboratory instruments, containers for cell and tissue culture and regeneration, supports for peptides, proteins and antibodies.

Accordingly, in a tenth aspect the present invention provides a medical device coated with a fraction of plasma and/or serum and at least one metal, metal ion or salt thereof, wherein said fraction is capable of healing tissue disruptions.

It is further contemplated that a therapeutic composition and/or wound dressing of the present invention may further comprise compounds, including but not limited to anti-microbials, anti-virals, growth factors, anti-dehydration compounds, coagulant agents such as Factor Xa, anti-septics, or other compounds suitable for biomedical and/or veterinary uses.

### BRIEF DESCRIPTION OF THE FIGURES

FIG. 1 shows regions of human skin exposed to UV light treated with the therapeutic composition of the present invention as compared to untreated skin.

FIG. 2 shows the same skin as in FIG. 1, but 7 weeks post-exposure.

### DETAILED DESCRIPTION OF THE INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional chemistry and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); The Merck Index, 12th Edition (1996), Therapeutic Category and Biological Activity Index; and Remington's Pharmaceutical Sciences, 17^{th} Edition, Mack Publishing Company, Easton, Pa., USA.

It must be noted that as used herein and in the appended claims, the singular forms “a,” “an,” and “the” include plural reference unless the context clearly dictates otherwise. Thus, for example, a reference to “a metal” includes a plurality of such metals, and a reference to “an isolated protein” is a reference to one or more proteins, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In its broadest aspect, the present invention provides a composition useful as a treatment agent for tissue disruptions. The term “tissue disruption” refers to abnormal conditions affecting animals, including humans, which can be treated using the agents, therapeutic compositions and wound dressings of the present invention. The term “tissue disruption” can include inflammation, lesions, wounds, soft tissue damage, connective tissue injury, non-air exposed injuries, such as bruises and deep soft tissue injuries such as tendon/ligament injuries, burns including all types of sun damage like sunburn and overuse injuries is included in the present invention.

The injury can be a minor tissue disruption of, for instance, epidermal, dermal, muscular or adipoidal tissue to the air. The term “wounds” includes a puncture wound, an incision, a laceration, a penetrating wound, a perforating wound, a tunnel wound and the like. Wounds also include open wounds that have been sutured or otherwise mechanically closed but have not healed or repaired the break in the skin or oral mucosal layer or of the surface layers of the eye including the conjunctiva and cornea.

The terms “lesion” and “surface lesion” as used herein refer to a circumscribed area of pathologically altered tissue, an injury or wound. Primary lesions are the immediate result of the pathological condition and include, but are not limited to, cuts, abrasions, vesicles, blebs, bullae chancres, pustules, tubercles or any other such condition of the skin or a surface of the mouth, nose, anus or any other orifice of the body of a human or animal, or to the surface layers of the eye including the conjunctiva and cornea, or secondary lesions that later develop from a primary lesion and includes, but is not limited to, fissures and ulcers and other wounds.

The term “tissue disruption management” refers to therapeutic methods that induce and/or promote repair of a tissue damage including, but not limited to, arresting tissue damage such as necrotization, promoting tissue growth and repair, reduction or elimination of an established microbial infection of the injury and prevention of new or additional microbial infection or colonization. The term may further include reducing or eliminating the sensation of pain attributable to a wound.

The terms “tissue disruption healing” and “tissue disruption repair” refer to a process involving tissue growth that partially or totally repairs the injury, repairs a breach in the dermis or epidermis and partially or totally restores the barrier properties of the skin or the repair of the surface layers of the eye including the conjunctiva and cornea.

Generally, the terms “treating,” “treatment” and the like are used herein to mean affecting an individual or animal, their tissue or cells to obtain a desired pharmacological and/or physiological effect. The effect is especially therapeutic in terms of a partial or complete cure of a condition such as a tissue disruption. “Treating” as used herein covers any treatment of a tissue disruption in a vertebrate, a mammal, particularly a human, and includes: (a) inhibiting the tissue disruption, i.e., arresting its development; or (b) relieving or ameliorating the symptoms of the tissue disruption, i.e., cause regression of the symptoms of the tissue disruption.

The terms “subject” or “individual” are used interchangeably herein to refer to any member of the class mammalia, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age. Thus, both adult and newborn individuals are intended to be covered.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses.

The term “effective amount” refers to that amount which is sufficient to induce tissue disruption healing or repair when administered to a subject; i.e., a tissue disruption healing amount. What constitutes an effective tissue disruption injury healing amount, or dose, of the composition of the present invention depends, among other factors, on the body weight of the subject and the degree of injury being treated. Normally an effective dose will be found in the range of about 1 to about 6 mg/kg body weight. For an average 75 kg subject, this range equates to a dose of about 75 to about 450 mg. Proportionately smaller or larger doses can be appropriate for subjects having lesser or greater body weight. Such a dose can be administered as needed, but typically administration 1 to about 4 times per day, in most cases 1 or 2 times a day, provides adequate tissue disruption healing.

The composition of the present invention essentially comprises a mixture of plasma and/or serum and at least one metal, metal ion or metal salt.

The terms “plasma” and “serum” are used herein interchangeably; however, the term “plasma” typically refers to the straw-coloured fluid in which the blood cells are suspended. It consists of various inorganic salts of sodium, potassium, calcium etc. with a high concentration of protein (approximately 70 g/l) and a variety of trace elements. The term “serum” refers to the fluid that separates from clotted blood or blood plasma that is allowed to stand. Serum is essentially similar in composition to plasma, but generally lacks fibrinogen and others substances that are used in the coagulation process.

The plasma or serum used in the present invention may be obtained from any animal source. Preferably, the plasma and/or serum is isolated from blood taken from an animal selected from the group consisting of human, equine, bovine, ovine, murine, caprine and canine.

In some embodiments, the animal source for the plasma or serum is bovine.

The plasma or serum may be freshly isolated or alternatively lyophilised. In one embodiment, blood is isolated from cattle and the haemoglobin is removed by standard procedures. The plasma is then preferably mixed with sodium bicarbonate (approx. 20 g per litre) and heated to about 80° C. The coagulated plasma protein is then removed and lyophilised by standard procedures for further use.

In some embodiments the lyophilised plasma or serum is resuspended in water (approximately 50 g per litre) and mixed with at least one metal.

Various metals and/or metal ions are useful in the composition of the present invention and as such the present invention embraces all such metals or metal ions.

In some embodiments, the metals are selected from the group consisting of nickel, sodium, copper, zinc, cobalt, iron, magnesium, manganese, potassium, silver and mercury.

In cases where the metals are sufficiently basic or acidic to form stable non-toxic acid or base salts, the use of the metals as salts can be appropriate. Examples of acceptable metal salts include acetate, ascorbate, benzoate, bicarbonate, chloride, citrate, carbonate, α-glycerophosphate, α-ketoglutarate, malonate, methanesulfonate, nitrate, succinate, sulfate, tartarate and tosylate salts.

Metal salts can be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts can be made.

In some embodiments, for example, the metal is silver (I), wherein the nitrate salt provides adequate free silver (I) ion to provide the necessary metal requirement. The chloride salt on the other hand provides less silver, being less soluble and with a low dissociation constant and therefore is less useful in the present invention. The skilled artisan will be able to readily determine the suitable salt form of the metal ion that provides the necessary properties for the present invention. Furthermore, the skilled artisan will be aware of the compatibility of the salt forms of the metal(s) and other components of the composition to maintain adequate levels of the metal ion(s).

In some embodiments, the metals used in the composition comprise a mixture of a number of metals. For example, the mixture of metals could consist essentially of NiSO₄.7H₂O, NH₄VO₃, NaF, CuSO₄.5H₂O, ZnCl₂, (NH₄)₆MO₇O₂₄.4H₂O, COCl₂.6H₂O, FeSO₄.7H₂O, MgSO₄.7H₂O, H₃BO₃, MnCl₂.4H₂O and K₂CrO₄.

Once the metal, metal ion or metal salt thereof has been mixed with the plasma and/or serum, it is preferably heated to at least 50° C. Preferably, the mixture is heated to about 65° C.

In some embodiments, a protease selected from the group consisting of trypsin, chymotrypsin, factor Xa, venom-protease, thrombin, plasmin and a serine-protease of the subtilisin family is preferably added before heating or after heating. Preferably, the protease is trypsin.

The protease can indeed be added before the metal, metal ion or metal salt is added. Whichever, once the protease has been added the resulting mixture of plasma/serum and protease, with or without metal, metal ion or metal salt is incubated between about 30° C. and 45° C. for at least 30 minutes. The mixture is then heated again. The second heating step is preferably carried out between about 80° C. and about 150° C., more preferably between about 90° C. and about 130° C. and most preferably, about 120° C. to produce said tissue disruption treatment mixture.

Once the tissue disruption treatment mixture has been obtained it can be either used directly or fractionated to obtain a tissue disruption treatment active fraction. Techniques for fractionating protein-containing mixtures are well known in the art. See, for example, “Plasma Protein Fractionation” Heide K, Haupt H & Schwick H; in The Plasma Proteins, 2nd Edition Vol 3 (1977) Putnam F. (Ed); U.S. Pat. No. 4,351,710 and U.S. Pat. No. 4,322,275 both entitled “Fractionation of protein mixtures”; U.S. Pat. No. 5,138,034 entitled “Method of fractionating plasma proteins” all incorporated herein by reference.

As described above, in one embodiment, the present invention provides a method of treating tissue disruptions in a subject, the method comprising administering to the subject an effective tissue disruption healing amount of a composition of the present invention.

The method of the invention can be used to treat all types of tissue disruptions as described supra. The method of the invention is useful for treatment of non-human mammalian subjects or patients, including domestic, farm and exotic animals, such as for example dogs horses, zoo animals and the like, but is primarily useful for treatment of human subjects or patients.

Compositions of the present invention can also be used in combination therapies with opioids and other analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods of the invention with one or more compounds selected from aceclofenac, acemetacin, α-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Still other suitable formulations for use in the present invention can be found in Remington's Pharmaceutical Sciences, Mace Publishing Company, Philadelphia, Pa. 17th ed. (1985).

The terms “administration,” administering,” and “administered” are used herein interchangeably. The tissue disruption healing composition of the present invention may be administered orally including sublingual, topically, or parenterally in dosage unit formulations containing conventional non-toxic pharmaceutically acceptable carriers, adjuvants, and vehicles. The term “parenteral” as used herein includes subcutaneous injections, aerosol, intravenous, intramuscular, intrathecal, intracranial, injection or infusion techniques or rectal or vaginally. Preferably, the tissue disruption healing composition of the present invention is administered together with a pharmaceutically acceptable carrier or diluent compatible with the composition. In preparing such composition, any conventional pharmaceutically acceptable carrier can be utilised.

The carrier material can be organic or inorganic inert carrier material suitable for oral administration. Suitable carriers include water, gelatin, gum arabic, lactose, starch, magnesium stearate, talc, vegetable oils, polyalkylene-glycols, petroleum jelly and the like. Furthermore, the pharmaceutically active preparations may contain other pharmaceutically active agents. Additionally, additives such as flavouring agents, preservatives, stabilisers, emulsifying agents, buffers and the like may be added in accordance with accepted practices of pharmaceutical compounding.

For topical administration to the skin or mucous membrane the aforementioned tissue disruption healing composition of the present invention is preferably prepared as an ointment, tincture, cream, gel, solution, lotion, spray; aerosol and dry powder for inhalation, suspension and the like. In fact, any conventional methods of preparing topical compositions can be utilised in this invention. Among the preferred methods of applying the tissue disruption healing composition of the present invention is in the form of an ointment, gel, cream, lotion, spray; aerosol or dry powder for inhalation. A pharmaceutical preparation for topical administration to the skin can be prepared by mixing the tissue disruption healing composition of the present invention with non-toxic, therapeutically inert, solid or liquid carriers customarily used in such preparation. These preparations generally contain 0.01 to 5.0 percent by weight, preferably 0.1 to 1.0 percent by weight, of the tissue disruption healing composition of the present invention, based on the total weight of the peptide preparation.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the afore-mentioned active agent. Among the conventional antioxidants which can be utilised in these preparations are included N-methyl-α-tocopherolamine, tocopherols, butylated hydroxyanisole, butylated hydroxytoluene, ethoxyquin and the like. Cream-base pharmaceutical formulations containing the antigen preparation, used in accordance with this invention, are composed of aqueous emulsions containing a fatty acid alcohol, semi-solid petroleum hydrocarbon, ethylene glycol and an emulsifying agent.

Ointment formulations containing the tissue disruption healing composition of the present invention comprise admixtures of a semi-solid petroleum hydrocarbon with a solvent dispersion of the tissue disruption healing composition. Cream compositions containing the tissue disruption healing composition of this invention preferably comprise emulsions formed from a water phase of a humectant, a viscosity stabiliser and water, an oil phase of a fatty acid alcohol, a semi-solid petroleum hydrocarbon and an emulsifying agent and a phase containing tissue disruption healing composition dispersed in an aqueous stabiliser-buffer solution. Stabilisers may be added to the topical preparation. Any conventional stabiliser can be utilised in accordance with this invention. In the oil phase, fatty acid alcohol components function as a stabiliser. These fatty acid alcohol components function as a stabiliser. These fatty acid alcohol components are derived from the reduction of a long-chain saturated fatty acid containing at least 14 carbon atoms.

Formulations for aerosols are described in Drugs and Pharmaceutical Sciences, Marcel Dekker, New York, 72: 547-574 (1996). Furthermore, the tissue disruption healing composition of the present invention can be delivered by dry powder inhalation. Such formulations and devices are described in Pharmaceutical Technology, June 1997, pp. 117-125.

Depending upon the mode or type of administration and the severity of the tissue disruption, the treatment regime will vary.

In one preferred embodiment, the compositions of the present invention are used directly as wound dressings. For example, as described supra, the resultant compositions can be used as a wound dressings directly. However, in a further embodiment the compositions of the present invention can be incorporated into “traditional” wound dressings such as plasters, bandages, gauze or pads.

In use, the wound dressings of the present invention are preferably used as the primary dressing placed in direct contact with the wound bed, or as near as practical against the wound bed. The dressings may serve as a packing material and, if required, may be secured into position with any suitable secondary wound dressing such as a wrap, tape, gauze, or pad. The dressings are temporary, however, and are not intended for permanent incorporation into the healed tissues. When necessary, the wound dressings are changed by first removing any over-dressing material and then removing the dressing, whereby any accumulated necrotic tissue and exudate is lifted away. The wound dressing of the present invention may be replaced by a fresh dressing or other suitable wound covering.

The dressings may be placed in their entirety into a wound. The dressings of the present invention may be cut, shaped and modified to accommodate numerous uses and applications.

A further use for the therapeutic composition of the present invention is in the delivery of therapeutically active agents including in any of the aforementioned applications. Therapeutically active agents may participate in, and improve, the healing process, and may include anti-microbial agents, including but not limited to anti-fungal agents, anti-bacterial agents, anti-viral agents and anti-parasitic agents, growth factors, angiogenic factors, anaesthetics, mucopolysaccharides, metals and other healing agents.

Examples of anti-microbial agents that can be used in the present invention include, but are not limited to, isoniazid, ethambutol, pyrazinamnide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, heavy metals including, but not limited to, gold, platinum, silver, zinc and copper, and their combined forms including, salts, such as chloride, bromide, iodide and periodate, and complexes with carriers, and other forms.

Growth factor agents that may be incorporated into the tissue disruption/wound dressing devices of the present invention include, but are not limited to, basic fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β) interleukin-8 (IL-8); granulocyte-macrophage colony stimulating factor (GM-CSF); the interleukins, and the interferons.

Other agents that may be incorporated into the dressings of the present invention are acid mucopolysaccharides including, but are not limited to, heparin, heparin sulfate, heparinoids, dermatan sulfate, pentosan polysulfate, cellulose, agarose, chitin, dextran, carrageenin, linoleic acid, and allantoin.

In some particularly preferred embodiments, the therapeutic composition of the present invention is admixed with coagulant agents such as Factor Xa.

The therapeutically active agents may be bound, either physically or chemically, to the therapeutic composition by methods well known in the art.

Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of specific animal plasma and metals, it will be clearly understood that the findings herein are not limited to these ingredients.

### Example 1

### Preparation of Tissue Disruption Treatment Composition

200 litres of sterile cattle blood was centrifuged at 1000-1300×g for 10 minutes and the haemoglobin was removed from the plasma. After centrifugation approximately 100 litres of plasma was gained, and transferred into a dish, suitable for heating and continuous mixing. To the plasma liquid 2 kg Sodium Bicarbonate (NaHCO₃) was added and mixed until the NaHCO₃ dissolved, then the solution was heated to 80° C. Denatured plasma protein was then recovered and placed on filter paper to dry. The solid sediment was then pressed to produce a 60 kg solid plasma-protein “block” which was then lyophilised by standard procedures. After this process the plasma-protein weighed approximately 8 kg and was used in the preparation of the tissue disruption treatment composition as described below.

A solution was then prepared comprising 152 litres of water, 8 kg dried plasma-protein as prepared above and 200 ml of a metal-containing solution. The constituents of the metal-containing solution are shown in Table 1.

TABLE 1
METAL-CONTAINING SOLUTION

| | |
|---|---|
| Ni SO₄ 7 h₂O | 10.4 g/l |
| NH₄VO₃ | 1.2 g/l |
| Na F | 24.0 g/l |
| Cu SO₄ 5H₂O | 20.0 g/l |
| ZN Cl₂ | 47.0 g/l |
| (NH₄) 6 MO₇O₂₄ 4H₂O | 7.0 g/l |
| CO Cl₂ 6H₂O | 20.0 g/l |
| Fe SO₄ 7H₂O | 100.0 g/l |
| MgSO₄ 7H₂O | 80.0 g/l |
| H₃BO₃ | 23.0 g/l |
| Glucose | 50.0 g/l |
| Mn Cl₂ 4H₂O | 36.4 g/l |
| K₂CrO₄ | 1.0 g/l |
| Glycine | 75.0 g/l |
| Citric Acid | 20.0 g/l |

Made up in a 200 ml solution with water, which was then stirred for at least 20 minutes.

The mixture was then heated up to 120° C. and maintained for two hours with constant mixing. During this time the plasma-protein dissolved and was sterilized. The resulting material was then held at a temperature of about 35° C. and 0.125 g/1 of trypsin was added. The material was then allowed to incubate for approximately 2 hours. The digested material was then autoclaved and cooled to produce the tissue disruption treatment composition of the present invention.

### Example 2

### Manufacture of a Topical Treatment Composition

A composition comprising the ingredients shown in Table 2 were mixed at 75-80° C. in a 250 litre vacuum homogenizer equipped with anchor and turbo mixers. Then the ingredients shown in Table 3 were added and the mixing was continued at 80-83° C. for 10 minutes with the aid of the turbo mixer.

A slow cooling process was then carried out using the anchor mixer. When the material reached 60° C., the vacuum was switched on until the end of the cooling.

At 40-45° C. the ingredients shown in Table 4 were added and mixed for 10 minutes. Mixing with the anchor mixer was continued until the mixture reached 25° C.

After a standing period of approximately 24 hours, the tissue disruption treatment composition was ready for use.

TABLE 2

| Item No. | Amount Per Kg | Ingredients |
|---|---|---|
| 1 | 20 g | Liposorb S20 (Tween 60) |
| 2 | 20 g | Cremaphor A6 |
| 3 | 10 g | Hydromyristenol |
| 4 | 40 g | Cetyl alcohol |
| 5 | 70 g | Corn Oil (Cold Pressed) |
| 6 | 30 g | Wheat Germ Oil |
| 7 | 0.24 g | Carrot Oil |
| 8 | 50 g | Isopropyl Myristate |
| 9 | 0.2 g | Butylated Hydroxytoluene B.P. |
| 10 | 3 g | Phenonip |

TABLE 3

| | | | |
|---|---|---|---|
| 11 | 400 | g | Plasma protein from Example 1 |
| 12 | 15 | g | Propylene Glycol B.P. |
| 13 | 15 | g | Hygroplex HHG |
| 14 | 2 | g | Allantoin |
| 15 | 208 | g | Purified Water B.P. |
| 16 | 10 | g | Germaben II |
| 17 | 4 | g | Veegum |
| 18 | 100 | g | Purified Water B.P. |
| 19 | 0.04 | ml | Potassium Bromide 50 g/l |
| 20 | 30.7 | mg | Sodium Sulphide |
| 21 | 0.04 | ml | Potassium Iodide 25 g/l |

TABLE 4

| | | |
|---|---|---|
| 22 | 1.4 g | Chammomile Fragrence |

### Methodology

1). Add items 1 to 10 in a 250 litre steam pan and heat 75° C.;
2). Boil items 15 and 18 in the 150 litre pan and transfer 13 litres to the 50 litre pan and add Veegum and mix until homogeneous;
3). Add item 14 to the remainder of the Purified Water B.P. in the 150 litre steam pan at above 90° C. and mix. When dissolved add the items 12, 13 and 16 and maintain temperature at 75° C. with continual mixing;
4). Add the water phase (step 5) to the oil phase (step 3) and mix using a short shaft air mixer. Then add step 4 to this using a plastic sieve to ensure that no lumps are incorporated;
5). Add plasma protein from Example 1 and emulsify for 20 minutes, then continue stirring whilst water cooling to 40° C.;
6). Add items 19 to 21 allowing a few minutes in between each addition whilst mixing. Cool to below 30° C.

### Example 3

### Clinical Trial on Topical Soft Tissue Injury Treatment

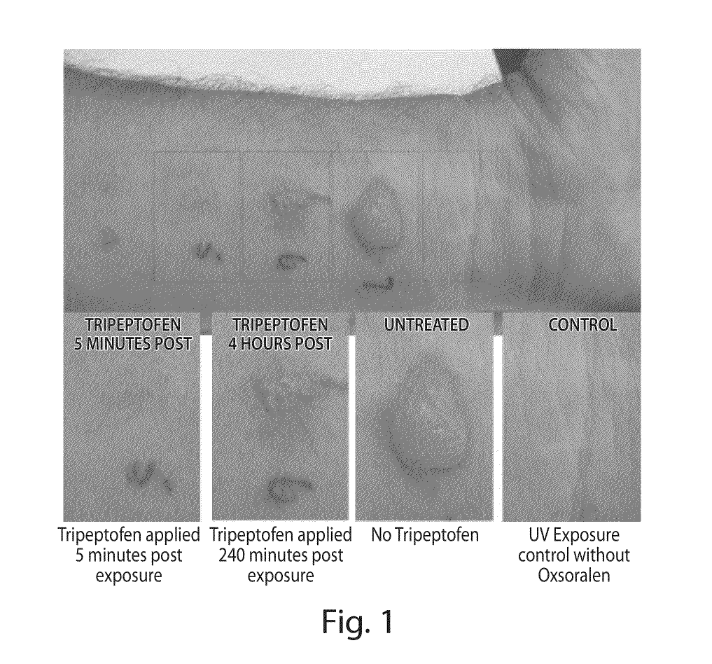

As shown in FIG. 1, a patient was exposed to UV light at 800 mJ for 10 minutes. Topical application of a 1% Oxsoralen (C₁₂H₈O₄) lotion was used on regions 5, 6 and 7 as a photo sensitizer. Region 8 remained an exposure control. Region 7 received no therapeutic treatment post exposure. Region 6 received topical treatment with the tissue disruption treatment composition described in Example 2 above after 240 minutes post-exposure, while region 5 received a similar amount of tissue disruption treatment composition 5 minutes post-exposure.

The pictures shown in FIG. 1 were taken 64 hours after exposure. As can be clearly seen there is a vast difference in both treated and untreated areas and between 5 minute post-exposure treatment and 240 minute post-exposure treatment with the tissue disruption treatment composition. As can be seen, control region 7 has a large fluid filled lesion after 64 hours. The application of the tissue disruption treatment composition after 240 minute post-exposure (Region 6) reduced the severity of the lesion produced. The application of the tissue disruption treatment composition to region 5 within 5 minutes of exposure appeared to protect the skin from lesion.

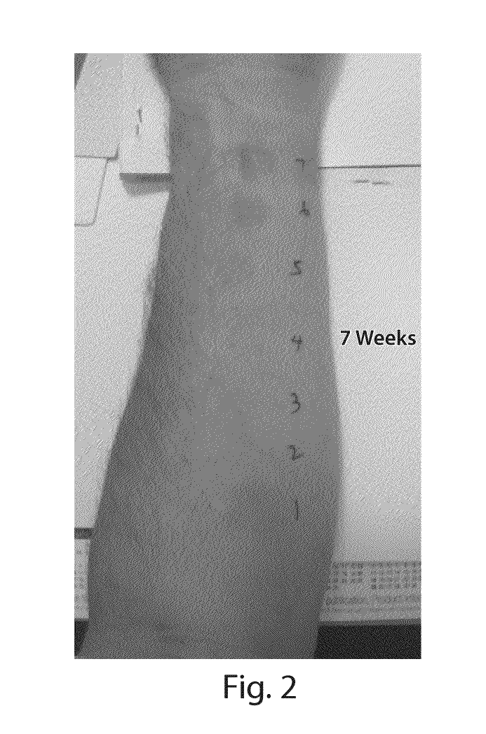

FIG. 2 shows the above regions 7 weeks post exposure. Apart from regions 1, 6 and 7 all of the regions had returned to normal skin.

1. A composition comprising an effective amount of an active fraction separated from a mixture of plasma and/or serum and at least one metal, metal ion or metal salt thereof, wherein said mixture has been denatured and is effective in healing tissue disruptions.

2. The composition according to claim 1, wherein the plasma or serum is isolated from an animal selected from the group consisting of human, equine, bovine, ovine, murine, caprine and canine.

3. A method of producing a composition obtained by:
a) heat denaturing a mixture of plasma and/or serum and at least one metal, metal ion or metal salt thereof; and
b) separating an active fraction from said denatured mixture;
wherein said active fraction is capable of healing tissue disruptions.

4. (canceled)

5. The method according to claim 3, further comprising the steps of incubating said mixture in the presence of a protease to produce a digested mixture; and heating said digested mixture.

6.-8. (canceled)

9. The method according to claim 3, wherein the metal is selected from the group consisting of nickel, sodium, copper, zinc, cobalt, iron, magnesium, manganese, potassium, silver and mercury, ions or salts thereof and mixtures thereof.

10. The method according to claim 3, wherein the metal is a mixture of metals consisting essentially of NiSO₄.7H₂O, NH₄VO₃, NaF, CuSO₄.5H₂O, ZnCl₂, (NH₄)₆Mo₇O₂₄.4H₂O, CoCl₂.6H₂O, FeSO₄.7H₂O, MgSO₄.7H₂O, H₃BO₃, MnCl₂.4H₂O and K₂CrO₄.

11. The method according to claim 3, wherein the step of heat denaturation is at a temperature of at least 50° C.

12. (canceled)

13. The method according to claim 3, wherein a protease is added before heating or after heating.

14. The method according to claim 13, wherein the protease is selected from the group consisting of trypsin, chymotrypsin, factor Xa, venom-protease, thrombin, plasmin and a serine-protease of the subtilisin family.

15. (canceled)

16. The method according to claim 13, wherein the mixture is further heated after addition of trypsin.

17.-20. (canceled)

21. The composition according to claim 1, optionally admixed with a pharmaceutical carrier.

22. (canceled)

23. The composition according to claim 1, further comprising a coagulation agent.

24. (canceled)

25. A composition obtained by:
(a) heat denaturing a mixture of plasma and/or serum and at least one metal, metal ion or metal salt thereof;
(b) incubating said mixture in the presence of a protease to produce a digested mixture;
(c) heating said digested mixture; and
(d) separating an active fraction from said denatured mixture;
wherein said active fraction is capable of healing tissue disruptions.

26. (canceled)

27. The composition according to claim 25, wherein steps (b) and (c) are performed before the addition of the at least one metal, metal ion or metal salt thereof.

28. The composition according to claim 25, wherein step (a) further comprises the addition of NaHCO₃.

29. (canceled)

30. A method for providing treatment of a tissue disruption in a subject, said method comprising administering to the subject an effective amount of a composition comprising an effective amount of an active fraction separated from a mixture of plasma and/or serum and at least one metal, metal ion or metal salt thereof, wherein said mixture has been denatured and wherein said active fraction is capable of healing tissue disruptions.

31. The method according to claim 30, wherein the subject is a human, an equine, a bovine, an ovine, a feline or a canine.

32.-33. (canceled)

33. (canceled)

34.-44. (canceled)

45. A method of treating a soft or connective tissue injury comprising the step of applying to said soft or connective tissue a therapeutic amount of a composition comprising an active fraction separated from a mixture of plasma and/or serum and at least one metal, metal ion or salt thereof, wherein said mixture has been denatured and wherein said fraction is admixed with a pharmaceutically acceptable carrier.

46. The method according to claim 45, wherein the plasma or serum is isolated from an animal selected from the group consisting of human, equine, bovine, ovine, murine, caprine and canine.

47.-50. (canceled)

51. The method according to claim 30, wherein the tissue disruption is selected from the group consisting of a lesion, a wound, a microbial infection, a burn, an ulcer, a soft tissue injury, a connective tissue injury, inflammation, and a dermal condition.
